(19) United States
(12) Patent Application Publication
(10) Pub. No.: US 2024/0197592 A1
(43) Pub. Date: Jun. 20, 2024

(54) COMPOSITIONS AND METHODS FOR REMOVING COLOR FROM HAIR

(71) Applicant: L'Oreal, Paris (FR)

(72) Inventors: Matthew PROEFKE, Troy, MI (US); Sarah SPEER, Linden, NJ (US); Tathagata MUKHEREJEE, Union, NJ (US); Shahid NASEER, Old Bridge, NJ (US); Sivaramakrishnan MUTHUKRISHNAN, Bridgewater, NJ (US)

(21) Appl. No.: 18/084,404

(22) Filed: Dec. 19, 2022

(51) Int. Cl. A61K 8/362; A45D 19/00; A61K 8/365; A61K 8/41; A61K 8/44; A61K 8/46; A61Q 5/08

(52) U.S. Cl. CPC A61K 8/362; A45D 19/0066; A61K 8/365; A61K 8/41; A61K 8/44; A61K 8/46; A61Q 5/08

(57) ABSTRACT

The disclosure relates to compositions and methods for removing color from keratin fibers such as hair. The compositions comprise (a) at least one reducing agent chosen from glutaric acid, glyoxylic acid, thiolactic acid, thioglycolic acid, glycolic acid, citric acid, or combinations of two or more thereof, (b) at least one amino acid, (c) optionally, at least one amine compound, and (d) at least one solvent.

### TECHNICAL FIELD

The present disclosure relates to compositions and methods for removing color from keratin fibers, such as hair.

### BACKGROUND

Consumers often desire to change the color of their hair. For example, as a person ages, the hair follicle loses its natural pigment, resulting in grey, silver, or white hair. It is common for consumers to color their grey hairs, for example to match their natural hair color. Similarly, consumers may wish to change the color of their hair from that of their natural color. For example, consumers often wish to lighten or darken their hair color.

The process of altering the color of hair is complex, however, particularly when permanent (oxidative) hair dyes are used. In some cases, the process requires that the natural pigment (melanin) in the hair fiber be destroyed before dye molecules of the desired color are added. This process is typically used when an individual desires to change the color of their hair to a shade lighter than their natural color, by dissolving the melanin with an oxidizing agent before the oxidative dye molecules penetrate the hair fiber where they achieve the target shade by undergoing a chemical reaction with an oxidizing agent. If an individual wishes to change the color of their hair to a shade darker than their natural shade it may not be necessary to destroy the melanin in the hair fiber, but the oxidative dye must still undergo a reaction with an oxidizing agent to achieve the target shade.

However, if an individual who has previously changed the color of their hair with a first oxidative dye wishes to alter the color of their previously-colored hair to achieve a second color or shade, the first oxidative dye, which is not able to be washed out, must be neutralized so that it no longer imparts the first color to the hair fiber. Although there are known methods for removing the first color from the hair by neutralizing (reducing) the first oxidative dye molecules, these methods are not completely satisfactory because the first oxidative dye molecules can re-oxidize, thus interfering with the ability of the consumer to achieve the desired second color or shade. Other known methods for removing the first color from the hair use oxidizing agents, but these compositions are harsh and lead to damage to the hair causing the hair to be weak, brittle, and have an unhealthy appearance.

It has now been surprisingly discovered that a synergistic combination of components unexpectedly removes oxidative dyes from hair in a manner that also prevents the dyes from re-oxidizing. The combination of components therefore can be used to alter the overall color of the hair by removing all or substantially all of the previously-applied color, or to remove only certain colors or shades, in order to vary the color or tone of the hair without removing all color (e.g. to alter the color of the hair to a warmer or cooler tone).

### SUMMARY

The disclosure relates to compositions and methods that successfully remove color from the hair, in particular hair that has previously been dyed with an oxidative dye composition.

In various embodiments, the disclosure relates to compositions for treating keratin fibers such as hair, the composition comprising (a) at least one reducing agent chosen from glutaric acid, glyoxylic acid, thiolactic acid, thioglycolic acid, glycolic acid, citric acid, or combinations of two or more thereof, (b) at least one amino acid, (c) optionally, at least one amine compound, and (d) at least one solvent. The amino acid may, in various embodiments, be chosen from betaine, aspartic acid, glutamic acid, alanine, arginine, ornithine, citrulline, asparagine, carnitine, cysteine, glutamine, glycine, histidine, lysine, isoleucine, leucine, methionine, N-phenylalanine, proline, serine, taurine, threonine, tryptophan, tyrosine, valine, or combinations of two or more thereof. If present, the amine compound may, in various embodiments, comprise, consist essentially of, or consist of monoethanolamine. The solvent may be chosen from water and/or non-aqueous solvents. The compositions are useful for treating keratin fibers such as hair in order to remove artificial color from the hair, such as oxidative dyes.

In various embodiments, the total amount of reducing agents (a) in the composition can range from about 0.1% to about 20%, such as from about 1% to about 18%, from 2% to about 15%, or from about 4% to about 12% by weight, relative to the total weight of the composition. In some embodiments, the total amount of reducing agents (a) ranges from about 3% to about 7%, from about 4% to about 6%, or is about 5% by weight, relative to the total weight of the composition. In other embodiments, the total amount of reducing agents (a) ranges from about 8% to about 12%, from about 9% to about 11%, or is about 10% by weight, relative to the total weight of the composition. In some embodiments, the compositions are free or essentially free of reducing agents other than glutaric acid, glyoxylic acid, thiolactic acid, thioglycolic acid, glycolic acid, and/or citric acid. In some embodiments, the glutaric acid is α-ketoglutaric acid.

In various embodiments, the total amount of amino acids (b) may range from about 0.1% to about 10%, such as from about 0.5% to about 8%, from about 1% to about 6%, or from about 1% to about 5% by weight, relative to the total weight of the composition. Optionally, the amino acids may comprise, consist essentially of, or consist of proline, betaine, arginine, glycine, or a combination of two or more thereof.

In various embodiments, the compositions comprise at least one amine compound, which may be present in an amount ranging from about 0.1% to about 20%, such as from about 1% to about 20%, from about 2% to about 15%, or from about 3% to about 10% by weight, relative to the total weight of the composition. The amine compound may, in some embodiments, comprise, consist essentially of, or consist of monoethanolamine. In various embodiments, the compositions comprise monoethanolamine in an amount ranging from about 1% to about 12%, such as from about 1.5% to about 10%, from about 2% to about 8%, or from about 3% to about 7% by weight, relative to the total weight of the composition.

Optionally, the compositions may comprise one or more additional components, such as carboxylic acids and/or salts thereof, surfactants, thickening agents, clay compounds, fatty compounds, or the like. In various embodiments, the compositions are free or essentially free of various components such as cationic surfactants, sulfites, bisulfites, metabisulfites, persulfates, peroxides, and/or ammonia.

The compositions are typically acidic, for example may have a pH ranging from about 1 to about 6, such as from about 2 to about 5, from about 2.5 to about 5, from about 2.75 to about 4.75, or from about 3 to about 4.5.

In exemplary embodiments, the disclosure relates to compositions for removing color from hair, the compositions comprising: (a) at least one reducing agent chosen from glutaric acid, glyoxylic acid, thiolactic acid, thioglycolic acid, glycolic acid, citric acid, or combinations of two or more thereof, (b) at least one amino acid, (c) optionally, at least one amine compound, and (d) at least one solvent; where the composition further comprises at least one component chosen from thickening agents, fatty compounds, clay compounds, carboxylic acids, anionic surfactants, non-ionic surfactants, amphoteric/zwitterionic surfactants, or combinations thereof; where the composition has a pH ranging from about 1 to about 6; and optionally where the composition is free or substantially free of reducing agents other than glutaric acid, glyoxylic acid, thiolactic acid, thioglycolic acid, glycolic acid, and/or citric acid. In further exemplary embodiments, the disclosure relates to compositions for removing color from hair, the compositions comprising: (a) at least one reducing agent chosen from glutaric acid, glyoxylic acid, thiolactic acid, thioglycolic acid, glycolic acid, citric acid, or combinations of two or more thereof, (b) at least one amino acid, (c) at least one amine compound, and (d) at least one solvent; where the composition further comprises at least one component chosen from thickening agents, fatty compounds, clay compounds, carboxylic acids, anionic surfactants, non-ionic surfactants, amphoteric/zwitterionic surfactants, or combinations thereof; where the composition has a pH ranging from about 1 to about 6; and optionally where the composition is free or substantially free of reducing agents other than glutaric acid, glyoxylic acid, thiolactic acid, thioglycolic acid, glycolic acid, and/or citric acid. In still further exemplary embodiments, the disclosure relates to compositions for removing color from hair, the compositions comprising: (a) at least one reducing agent chosen from glutaric acid, glyoxylic acid, thiolactic acid, thioglycolic acid, glycolic acid, citric acid, or combinations of two or more thereof, (b) at least one amino acid, (c) monoethanolamine, and (d) at least one solvent; where the composition further comprises at least one component chosen from thickening agents, fatty compounds, clay compounds, carboxylic acids, anionic surfactants, non-ionic surfactants, amphoteric/zwitterionic surfactants, or combinations thereof; where the composition has a pH ranging from about 1 to about 6; and optionally where the composition is free or substantially free of reducing agents other than glutaric acid, glyoxylic acid, thiolactic acid, thioglycolic acid, glycolic acid, and/or citric acid.

In other exemplary embodiments, the disclosure relates to compositions for removing color from hair, the compositions comprising: (a) at least one reducing agent chosen from glutaric acid, glyoxylic acid, thiolactic acid, thioglycolic acid, glycolic acid, citric acid, or combinations of two or more thereof, (b) at least one amino acid chosen from proline, betaine, arginine, glycine, or a combination of two or more thereof, (c) optionally, at least one amine compound, and (d) at least one solvent; where the composition further comprises at least one component chosen from thickening agents, fatty compounds, clay compounds, carboxylic acids, anionic surfactants, non-ionic surfactants, amphoteric/zwitterionic surfactants, or combinations thereof; where the composition has a pH ranging from about 1 to about 6; and optionally where the composition is free or substantially free of reducing agents other than glutaric acid, glyoxylic acid, thiolactic acid, thioglycolic acid, glycolic acid, and/or citric acid. In further exemplary embodiments, the disclosure relates to compositions for removing color from hair, the compositions comprising: (a) at least one reducing agent chosen from glutaric acid, glyoxylic acid, thiolactic acid, thioglycolic acid, glycolic acid, citric acid, or combinations of two or more thereof, (b) at least one amino acid chosen from proline, betaine, arginine, glycine, or a combination of two or more thereof, (c) at least one amine compound, and (d) at least one solvent; where the composition further comprises at least one component chosen from thickening agents, fatty compounds, clay compounds, carboxylic acids, anionic surfactants, non-ionic surfactants, amphoteric/zwitterionic surfactants, or combinations thereof; where the composition has a pH ranging from about 1 to about 6; and optionally where the composition is free or substantially free of reducing agents other than glutaric acid, glyoxylic acid, thiolactic acid, thioglycolic acid, glycolic acid, and/or citric acid. In still further exemplary embodiments, the disclosure relates to compositions for removing color from hair, the compositions comprising: (a) at least one reducing agent chosen from glutaric acid, glyoxylic acid, thiolactic acid, thioglycolic acid, glycolic acid, citric acid, or combinations of two or more thereof, (b) at least one amino acid chosen from proline, betaine, arginine, glycine, or a combination of two or more thereof, (c) monoethanolamine, and (d) at least one solvent; where the composition further comprises at least one component chosen from thickening agents, fatty compounds, clay compounds, carboxylic acids, anionic surfactants, non-ionic surfactants, amphoteric/zwitterionic surfactants, or combinations thereof; where the composition has a pH ranging from about 1 to about 6; and optionally where the composition is free or substantially free of reducing agents other than glutaric acid, glyoxylic acid, thiolactic acid, thioglycolic acid, glycolic acid, and/or citric acid.

In additional exemplary embodiments, the disclosure relates to compositions for removing color from hair, the compositions comprising: (a) α-ketoglutaric acid, and optionally at least one additional reducing agent chosen from glutaric acid compounds other than α-ketoglutaric acid, glyoxylic acid, thiolactic acid, thioglycolic acid, glycolic acid, and/or citric acid, (b) at least one amino acid, (c) optionally, at least one amine compound, and (d) at least one solvent; where the composition further comprises at least one component chosen from thickening agents, fatty compounds, clay compounds, carboxylic acids, anionic surfactants, non-ionic surfactants, amphoteric/zwitterionic surfactants, or combinations thereof; where the composition has a pH ranging from about 1 to about 6; and optionally where the composition is free or substantially free of reducing agents other than glutaric acid, glyoxylic acid, thiolactic acid, thioglycolic acid, glycolic acid, and/or citric acid. In further exemplary embodiments, the disclosure relates to compositions for removing color from hair, the compositions comprising: (a) α-ketoglutaric acid, and optionally at least one additional reducing agent chosen from glutaric acid compounds other than α-ketoglutaric acid, glyoxylic acid, thiolactic acid, thioglycolic acid, glycolic acid, and/or citric acid, (b) at least one amino acid, (c) at least one amine compound, and (d) at least one solvent; where the composition further comprises at least one component chosen from thickening agents, fatty compounds, clay compounds, carboxylic acids, anionic surfactants, non-ionic surfactants, amphoteric/zwitterionic surfactants, or combinations thereof; where the composition has a pH ranging from about 1 to about 6; and optionally where the composition is free or substantially free of reducing agents other than glutaric acid, glyoxylic acid, thiolactic acid, thioglycolic acid, glycolic acid, and/or citric acid. In still further exemplary embodiments, the disclosure relates to compositions for removing color from hair, the compositions comprising: (a) α-ketoglutaric acid, and optionally at least one additional reducing agent chosen from glutaric acid compounds other than α-ketoglutaric acid, glyoxylic acid, thiolactic acid, thioglycolic acid, glycolic acid, and/or citric acid, (b) at least one amino acid, (c) monoethanolamine, and (d) at least one solvent; where the composition further comprises at least one component chosen from thickening agents, fatty compounds, clay compounds, carboxylic acids, anionic surfactants, non-ionic surfactants, amphoteric/zwitterionic surfactants, or combinations thereof; where the composition has a pH ranging from about 1 to about 6; and optionally where the composition is free or substantially free of reducing agents other than glutaric acid, glyoxylic acid, thiolactic acid, thioglycolic acid, glycolic acid, and/or citric acid.

In various embodiments, the disclosure relates to compositions for removing color from hair, the composition comprising (a) at least one reducing agent chosen from glutaric acid, glyoxylic acid, thiolactic acid, thioglycolic acid, glycolic acid, citric acid, or combinations of two or more thereof, (b) at least one amino acid, (c) monoethanolamine, and (d) water; where the composition optionally further comprises at least one component chosen from thickening agents, fatty compounds, clay compounds, carboxylic acids, anionic surfactants, non-ionic surfactants, amphoteric/zwitterionic surfactants, or combinations thereof; where the composition has a pH ranging from about 1 to about 6; and optionally where the composition is free or substantially free of reducing agents other than glutaric acid, glyoxylic acid, thiolactic acid, thioglycolic acid, glycolic acid, and/or citric acid.

In further embodiments, the disclosure relates to compositions for removing color from hair, the composition comprising (a) α-ketoglutaric acid, and optionally at least one additional reducing agent chosen from glutaric acid compounds other than α-ketoglutaric acid, glyoxylic acid, thiolactic acid, thioglycolic acid, glycolic acid, and/or citric acid, (b) at least one amino acid chosen from proline, betaine, arginine, glycine, or a combination of two or more thereof, (c) optionally monoethanolamine, and (d) at least one solvent; where the composition optionally further comprises at least one component chosen from thickening agents, fatty compounds, clay compounds, carboxylic acids, anionic surfactants, non-ionic surfactants, amphoteric/zwitterionic surfactants, or combinations thereof; where the composition has a pH ranging from about 1 to about 6; and where the composition is optionally free or substantially free of reducing agents other than glutaric acid, glyoxylic acid, thiolactic acid, thioglycolic acid, glycolic acid, and/or citric acid.

The disclosure also relates to methods of treating hair using the compositions according to the disclosure. For example, the methods may be methods of removing oxidative dyes from the hair. In some embodiments, a composition according to the disclosure may be applied to the hair and left on the hair for a period of time, for example up to about 2 hours, such as from about 30 seconds to about 2 hours, from about 1 minute to about 1 hour, from about 1 minute to about 45 minutes, from about 1 minute to about 30 minutes, from about 5 minutes to about 1 hour, from about 5 minutes to about 45 minutes, from about 5 minutes to about 30 minutes, etc. The methods may further comprise rinsing the hair.

### DETAILED DESCRIPTION

The disclosure relates to compositions and methods for removing color, in particular color imparted by previously-applied oxidative dyes, from the hair. The color-removal benefits according to various embodiments may be long-lasting, e.g. may last for at least 7 days, at least 1 month, at least 2 months, etc.

### I. Compositions

The compositions according to the disclosure comprise at least one reducing agent, at least one amino acid, at least one solvent, and optionally at least one amine compound.

### Reducing Agents

Compositions according to the disclosure comprise at least one reducing agent chosen from glutaric acid (which expressly includes the ketone derivatives thereof, α-ketoglutaric acid and β-ketoglutaric acid), glyoxylic acid, thiolactic acid, thioglycolic acid, glycolic acid, citric acid, or combinations of two or more thereof. These reducing agents have been found to be surprisingly effective at removing color from hair, even in the absence of additional components that may typically be included in hair color removal compositions.

In various embodiments, the total amount of reducing agents chosen from glutaric acid, glyoxylic acid, thiolactic acid, thioglycolic acid, glycolic acid, citric acid, or combinations of two or more thereof may range up to about 20%, such as from about 1% to about 15%, from about 2% to about 13%, from about 3% to about 12%, from about 4% to about 11%, or from about 5% to about 10% by weight, relative to the total weight of the composition. For example, the total amount of reducing agents chosen from glutaric acid, glyoxylic acid, thiolactic acid, thioglycolic acid, glycolic acid, citric acid, or combinations of two or more thereof may from about 1% to about 20%, from about 1% to about 15%, from about 1% to about 14%, from about 1% to about 13%, from about 1% to about 12%, from about 1% to about 11%, from about 1% to about 10%, from about 1% to about 9%, from about 1% to about 8%, from about 1% to about 7%, from about 1% to about 6%, from about 1% to about 5%, from about 2% to about 20%, from about 2% to about 15%, from about 2% to about 14%, from about 2% to about 13%, from about 2% to about 12%, from about 2% to about 11%, from about 2% to about 10%, from about 2% to about 9%, from about 2% to about 8%, from about 2% to about 7%, from about 2% to about 6%, from about 2% to about 5%, from about 3% to about 20%, from about 3% to about 15%, from about 3% to about 14%, from about 3% to about 13%, from about 3% to about 12%, from about 3% to about 11%, from about 3% to about 10%, from about 3% to about 9%, from about 3% to about 8%, from about 3% to about 7%, from about 3% to about 6%, from about 3% to about 5%, from about 4% to about 20%, from about 4% to about 15%, from about 4% to about 14%, from about 4% to about 13%, from about 4% to about 12%, from about 4% to about 11%, from about 4% to about 10%, from about 4% to about 9%, from about 4% to about 8%, from about 4% to about 7%, from about 4% to about 6%, from about 4% to about 5%, from about 5% to about 20%, from about 5% to about 15%, from about 5% to about 14%, from about 5% to about 13%, from about 5% to about 12%, from about 5% to about 11%, from about 5% to about 10%, from about 5% to about 9%, from about 5% to about 8%, from about 5% to about 7%, or from about 5% to about 6%, from about 6% to about 20%, from about 6% to about 15%, from about 6% to about 14%, from about 6% to about 13%, from about 6% to about 12%, from about 6% to about 11%, from about 6% to about 10%, from about 6% to about 9%, from about 6% to about 8%, from about 6% to about 7%, from about 7% to about 20%, from about 7% to about 15%, from about 7% to about 14%, from about 7% to about 13%, from about 7% to about 12%, from about 7% to about 11%, from about 7% to about 10%, from about 7% to about 9%, from about 7% to about 8%, from about 8% to about 20%, from about 8% to about 15%, from about 8% to about 14%, from about 8% to about 13%, from about 8% to about 12%, from about 8% to about 11%, from about 8% to about 10%, from about 8% to about 9%, from about 9% to about 20%, from about 9% to about 15%, from about 9% to about 14%, from about 9% to about 13%, from about 9% to about 12%, from about 9% to about 11%, from about 9% to about 10%, from about 10% to about 20%, from about 10% to about 15%, from about 10% to about 14%, from about 10% to about 13%, from about 10% to about 12%, or from about 10% to about 11% by weight, relative to the total weight of the composition. For example, the total amount of reducing agents chosen from glutaric acid, glyoxylic acid, thiolactic acid, thioglycolic acid, glycolic acid, citric acid, or combinations of two or more thereof may be about 3%, about 4%, about 5%, about 6%, about 7%, about 8%, about 9%, about 10%, about 11%, or about 12% by weight, relative to the total weight of the composition, including any range using any of the foregoing as upper and lower limits.

In some embodiments, the reducing agent comprises, consists essentially of, or consists of α-ketoglutaric acid.

In various embodiments, the compositions are free or essentially free of reducing agents other than glutaric acid, glyoxylic acid, thiolactic acid, thioglycolic acid, glycolic acid, and/or citric acid. For example, in some embodiments, the compositions may contain no more than about 20%, such as no more than about 15%, no more than about 10%, or no more than about 5% of other reducing agents. In other embodiments, if one or more reducing agents other than glutaric acid, glyoxylic acid, thiolactic acid, thioglycolic acid, glycolic acid, and/or citric acid are present, these other reducing agents are present in an amount that is not sufficient, by itself, to remove the color from the hair, e.g. less than about 3%, less than about 2.5%, less than about 2%, less than about 1.5%, less than about 1%, or less than about 0.5% by weight, relative to the total weight of the composition.

### Amino Acids

Compositions according to the disclosure comprise at least one amino acid. In some embodiments, compositions according to the disclosure can comprise at least one, at least two, or at least three amino acids.

Amino acids are simple organic compounds containing an amino group (—NH₂) and at least one acid function chosen from carboxylic acid, sulfonic acid, phosphonic acid, or phosphoric acid functions, along with a side chain (R group) specific to each amino acid. The amino acids may be of natural or synthetic origin, may be in their L-form, D-form, or a racemic mixture, and may be neutral or ionic.

Non-limiting examples of useful amino acids include aspartic acid, glutamic acid, alanine, arginine, ornithine, citrulline, asparagine, carnitine, cysteine, glutamine, glycine, histidine, lysine, isoleucine, leucine, methionine, N-phenylalanine, proline, serine, taurine, threonine, tryptophan, tyrosine, and valine. Derivatives of these amino acids may also be used.

For example, in various embodiments, the at least one amino acid may be chosen from amino acids such as arginine, histidine, lysine, glycine, methyl derivatives of glycine such as betaine (also referred to as trimethylglycine), proline, or combinations thereof. In some embodiments, the amino acids comprise, consist essentially of, or consist of proline, betaine, arginine, glycine, or a combination of two or more thereof.

The total amount of amino acids may range from about 0.1% to about 10%, from about 0.1% to about 9%, about 0.1% to about 8%, from about 0.1% to about 7%, from about 0.1% to about 6%, from about 0.1% to about 5%, from about 0.1% to about 4%, from about 0.1% to about 3%, from about 0.1% to about 2%, from about 0.1% to about 1%, from about 0.5% to about 10%, from about 0.5% to about 9%, from about 0.5% to about 8%, from about 0.5% to about 7%, from about 0.5% to about 6%, from about 0.5% to about 5%, from about 0.5% to about 4%, from about 0.5% to about 3%, from about 0.5% to about 2%, from about 0.5% to about 1%, from about 1% to about 10%, from about 1% to about 9%, from about 1% to about 8%, from about 1% to about 7%, from about 1% to about 6%, from about 1% to about 5%, from about 1% to about 4%, from about 1% to about 3%, from about 1% to about 2%, from about 1.5% to about 10%, from about 1.5% to about 9%, from about 1.5% to about 8%, from about 1.5% to about 7%, from about 1.5% to about 6%, from about 1.5% to about 5%, from about 1.5% to about 4%, from about 1.5% to about 3%, from about 1.5% to about 2%, by weight, relative to the total weight of the composition. In some embodiments, the total amount of amino acids ranges from about 1% to about 6%, from about 1% to about 5.5%, from about 1% to about 5%, from about 1% to about 4.5%, from about 1% to about 4%, from about 1% to about 3.5%, from about 1.5% to about 5%, from about 1.5% to about 4%, from about 1.5% to about 3.5%, or from about 1.5% to about 3% by weight, relative to the total weight of the composition.

In an exemplary embodiment, the amino acids comprise, consist essentially of, or consist of proline, betaine, arginine, glycine, or a combination of two or more thereof, in a total amount ranging from about 1% to about 6%, from about 1% to about 5.5%, from about 1% to about 5%, from about 1% to about 4.5%, from about 1% to about 4%, from about 1% to about 3.5%, from about 1.5% to about 5%, from about 1.5% to about 4%, from about 1.5% to about 3.5%, or about 1.5% to about 3% by weight, relative to the total weight of the composition.

### Solvents

Compositions according to the disclosure comprise at least one solvent. The solvent may be chosen from water, non-aqueous solvents, or combinations thereof.

In some embodiments, the solvent comprises, consists essentially of, or consists of water. The total amount of water may vary depending on the desired properties of composition, for example consistency, viscosity, etc.

In some embodiments, non-aqueous solvents maybe used, for example, glycerin, C₁₋₄ alcohols, fatty alcohols, fatty ethers, fatty esters, polyols, glycols, vegetable oils, mineral oils, liposomes, laminar lipid materials, or combinations thereof. Non-limiting examples of non-aqueous solvents include alkanediols such as glycerin, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, or combinations thereof.

Typically, the total amount of solvents in the composition may range from about 50% to about 98% by weight, such as from about 55% to about 95%, from about 60% to about 95%, from about 60% to about 90%, or from about 65% to about 90%, relative to the total weight of the composition.

### Amine Compounds

Compositions according to the disclosure may optionally also comprise at least one amine compound. It has been found that, at least in some instances, including amine compounds in the compositions may aid in the color removal process.

In various embodiments, the amine compounds can be chosen from alkanolamines. For example, monoethanolamine, diethanolamine, triethanolamine aminomethylpropanol, or the like may be added. In some embodiments, monoethanolamine works surprisingly synergistically with reducing agents according to the disclosure.

If present, the total amount of amine compounds present in the composition may range from about 0.1% to about 20%, such as from about 1% to about 18%, from about 3% to about 16%, or from about 5% to about 15% by weight, relative to the total weight of the composition. For example, the total amount of amine compounds may range from about 0.1% to about 9%, from about 0.1% to about 8%, from about 0.1% to about 7%, from about 0.1% to about 6%, from about 0.1% to about 5%, from about 0.1% to about 4%, from about 0.1% to about 3%, from about 0.1% to about 2%, from about 0.1% to about 1%, from about 0.5% to about 10%, from about 0.5% to about 9%, from about 0.5% to about 8%, from about 0.5% to about 7%, from about 0.5% to about 6%, from about 0.5% to about 5%, from about 0.5% to about 4%, from about 0.5% to about 3%, from about 0.5% to about 2%, from about 0.5% to about 1%, from about 1% to about 10%, from about 1% to about 9%, from about 1% to about 8%, from about 1% to about 7%, from about 1% to about 6%, from about 1% to about 5%, from about 1% to about 4%, from about 1% to about 3%, from about 1% to about 2%, from about 1.5% to about 10%, from about 1.5% to about 9%, from about 1.5% to about 8%, from about 1.5% to about 7%, from about 1.5% to about 6%, from about 1.5% to about 5%, from about 1.5% to about 4%, from about 1.5% to about 3%, from about 1.5% to about 2%, from about 2% to about 10%, from about 2% to about 9%, from about 2% to about 8%, from about 2% to about 7%, from about 2% to about 6%, from about 2% to about 5%, from about 2% to about 4%, from about 2% to about 3% by weight, relative to the total weight of the composition. In some embodiments, the total amount of amine compounds ranges from about 1.5% to about 5%, from about 1.5% to about 4%, from about 1.5% to about 3.5%, from about 2 to about 6%, about 2% to about 5%, or about 2% to about 4% by weight, relative to the total weight of the composition.

In some embodiments, the amine compound comprises, consists essentially of, or consists of alkanolamines, which may be present in a total amount ranging from about 1% to about 12%, such as from about 1.5% to about 10%, from about 2% to about 8%, or from about 3% to about 7% by weight, relative to the total weight of the composition. In some embodiments, the amine compound comprises, consists essentially of, or consists of alkanolamines, which may be present in a total amount of about 1%, about 2%, about 3%, about 4%, about 5%, about 6%, about 7%, about 8%, about 9%, or about 10% by weight, relative to the total weight of the composition, including any range using any of the foregoing as upper and lower limits.

In further embodiments, the amine compound comprises, consists essentially of, or consists of monoethanolamine, and the monoethanolamine may be present in an amount ranging from about 1% to about 12%, such as from about 1.5% to about 10%, from about 2% to about 8%, or from about 3% to about 7% by weight, relative to the total weight of the composition. In some embodiments, the amine compound comprises, consists essentially of, or consists of monoethanolamine, and the monoethanolamine may be present in an amount of about 1%, about 2%, about 3%, about 4%, about 5%, about 6%, about 7%, about 8%, about 9%, or about 10% by weight, relative to the total weight of the composition, including any range using any of the foregoing as upper and lower limits.

### Clay Compounds

Compositions according to the disclosure optionally comprise at least one clay compound. In some embodiments, the compositions comprise more than one clay compound, for example, at least two clay compounds, at least three clay compounds, etc. The clay compounds may, in some cases, provide a reduction in the odor which is typically associated with hair color removing compositions.

By way of non-limiting example, the clay compound may be chosen from kaolinite (also referred to interchangeably as kaolin), bentonite, smectite, hectorite, vermiculite, illite, chorite, halloysite, palygorskite, sepiolite, sesquioxide, imogolite, allophane, or combinations of two or more thereof. For example, in one embodiment, kaolin is chosen. In another embodiment, bentonite is chosen. In various embodiments, the clay compound comprises, consists essentially of, or consists of smectite, hectorite, kaolin, bentonite, or a combination of two or more thereof.

If present, the total amount of clay can range from about 0.1% to about 15%, such as from about 0.5% to about 12%, from about 1% to about 10%, or from about 5% to about 10% by weight, relative to the total weight of the composition. For example, the total amount of clay may range from about 1% to about 12%, from about 1% to about 11%, from about 1% to about 10%, from about 1% to about 9%, from about 1% to about 8%, from about 1% to about 7%, from about 1% to about 6%, from about 1% to about 5%, from about 1% to about 4%, from about 1% to about 3%, from about 1% to about 2%, from about 2% to about 12%, from about 2% to about 11%, from about 2% to about 10%, from about 2% to about 9%, from about 2% to about 8%, from about 2% to about 7%, from about 2% to about 6%, from about 2% to about 5%, from about 2% to about 4%, from about 2% to about 3%, from about 3% to about 12%, from about 3% to about 11%, from about 3% to about 10%, from about 3% to about 9%, from about 3% to about 8%, from about 3% to about 7%, from about 3% to about 6%, from about 3% to about 5%, from about 3% to about 4%, from about 4% to about 12%, from about 4% to about 11%, from about 4% to about 10%, from about 4% to about 9%, from about 4% to about 8%, from about 4% to about 7%, from about 4% to about 6%, from about 4% to about 5%, from about 5% to about 12%, from about 5% to about 11%, from about 5% to about 10%, from about 5% to about 9%, from about 5% to about 8%, from about 5% to about 7%, from about 5% to about 6%, from about 6% to about 12%, from about 6% to about 11%, from about 6% to about 10%, from about 6% to about 9%, from about 6% to about 8%, from about 6% to about 7%, from about 7% to about 12%, from about 7% to about 11%, from about 7% to about 10%, from about 7% to about 9%, from about 7% to about 8%, from about 8% to about 12%, from about 8% to about 11%, from about 8% to about 10%, from about 8% to about 9%, from about 9% to about 12%, from about 9% to about 11%, from about 9% to about 10%, from about 10% to about 12%, or from about 10% to about 11% by weight, relative to the total weight of the composition. For example, the total amount of clays may be about 3%, about 4%, about 5%, about 6%, about 7%, about 8%, about 9%, about 10%, about 11%, or about 12% by weight, relative to the total weight of the composition, including any range using any of the foregoing as upper and lower limits.

As a non-limiting example, the clay compound may comprise, consist essentially of, or consist of bentonite, kaolin, smectite, hectorite, or a combination of two or more thereof, and the total amount of clays may be about 3%, about 4%, about 5%, about 6%, about 7%, about 8%, about 9%, about 10%, about 11%, or about 12% by weight, relative to the total weight of the composition, or may be a range using any of the foregoing as upper or lower limits.

### Surfactants

Compositions according to the disclosure may optionally comprise at least one surfactant chosen from anionic, non-ionic, and/or amphoteric or zwitterionic surfactants. For example, the compositions may comprise one or more anionic surfactants, one or more non-ionic surfactants, or one or more amphoteric/zwitterionic surfactants, or the compositions may comprise mixtures of surfactants having the same or different ionicities.

### Anionic Surfactants

In at least some embodiments, the compositions comprise at least at least one anionic surfactant. The term “anionic surfactant” means a surfactant comprising, as ionic or ionizable groups, only anionic groups. A species is termed as being “anionic” when it bears at least one permanent negative charge or when it can be ionized as a negatively charged species, under the conditions of use of the composition (for example the medium or the pH) and not comprising any cationic charge. These anionic groups may be chosen from —CO₂H, —CO₂⁻, —SO₃H, —SO₃⁻, —OSO₃H, —OSO₃⁻, —H₂PO₃, —HPO₃⁻, —PO₃²⁻, —H₂PO₂, ═HPO₂, —HPO₂⁻, ═PO₂⁻, ═POH, and ═PO⁻ groups.

The anionic surfactants may be sulfate, sulfonate, and/or carboxylic (or carboxylate) surfactants, or mixtures thereof.

Sulfate anionic surfactants comprise at least one sulfate function but do not comprise any carboxylate or sulfonate functions. The sulfate anionic surfactants that may be used comprise at least one sulfate function (—OSO₃H or —OSO₃).

Non-limiting examples of sulfate anionic surfactants include alkyl sulfates, alkyl ether sulfates, alkylamido ether sulfates, alkylaryl polyether sulfates, monoglyceride sulfates; and also the salts of these compounds; the alkyl groups of these compounds comprising from 6 to 30 carbon atoms, especially from 12 to 28, better still from 14 to 24 or even from 16 to 22 carbon atoms; the aryl group preferably denoting a phenyl or benzyl group; these compounds possibly being polyoxyalkylenated, especially polyoxyethylenated, and then preferably comprising from 1 to 50 ethylene oxide units and better still from 2 to 10 ethylene oxide units.

Sulfonate anionic surfactants comprise at least one sulfonate function (—SO₃H or —SO₃⁻) and may optionally also comprise one or more sulfate functions, but do not comprise any carboxylate functions. The sulfonate anionic surfactants that may be used comprise at least one sulfonate function (—SO₃H or —SO₃⁻).

Non-limiting examples of sulfonate anionic surfactants include alkylsulfonates, alkylamidesulfonates, alkylarylsulfonates, α-olefinsulfonates, paraffin sulfonates, alkylsulfosuccinates, alkyl ether sulfosuccinates, alkylamidesulfosuccinates, alkylsulfoacetates, N-acyltaurates, acylisethionates; alkylsulfolaurates; and also the salts of these compounds; the alkyl groups of these compounds comprising from 6 to 30 carbon atoms, especially from 12 to 28, better still from 14 to 24 or even from 16 to 22 carbon atoms; the aryl group preferably denoting a phenyl or benzyl group; these compounds possibly being polyoxyalkylenated, especially polyoxyethylenated, and then preferably comprising from 1 to 50 ethylene oxide units and better still from 2 to 10 ethylene oxide units.

Carboxylate anionic surfactants comprise at least one carboxylic or carboxylate function (—COOH or —COO⁻) and may optionally also comprise one or more sulfate and/or sulfonate functions. The carboxylic anionic surfactants that may be used thus comprise at least one carboxylic or carboxylate function (—OOH or —COO⁻).

Non-limiting examples of carboxylate anionic surfactants include acylglycinates, acyllactylates, acylsarcosinates, acylglutamates, alkyl-D-galactosideuronic acids, alkyl ether carboxylic acids, alkyl(C6-30 aryl) ether carboxylic acids, alkylamido ether carboxylic acids; and also the salts of these compounds; the alkyl and/or acyl groups of these compounds comprising from 6 to 30 carbon atoms, especially from 12 to 28, better still from 14 to 24 or even from 16 to 22 carbon atoms; the aryl group preferably denoting a phenyl or benzyl group; these compounds possibly being polyoxyalkylenated, especially polyoxyethylenated, and then preferably comprising from 1 to 50 ethylene oxide units and better still from 2 to 10 ethylene oxide units.

When the anionic surfactant is in salt form, the salt may be chosen from alkali metal salts, such as the sodium or potassium salt, ammonium salts, amine salts and in particular amino alcohol salts, and alkaline-earth metal salts, such as the magnesium salt.

Examples of amino alcohol salts may include but are not limited to monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts.

In some embodiments, alkali metal or alkaline-earth metal salts and in particular the sodium or magnesium salts may be chosen, especially in the form of alkali metal, ammonium, amino alcohol and alkaline-earth metal salts, or a mixture of these compounds, may be chosen.

In exemplary and non-limiting embodiments, the anionic surfactant may be chosen from sodium laureth sulfate, ammonium laureth sulfate, disodium lauryl sulfosuccinate, disodium laureth sulfosuccinate, diammonium lauryl sulfosuccinate, diethylhexyl sodium sulfosuccinate, sodium oleyl succinate, sodium lauroyl methyl isethionate, sodium lauryl isethionate, sodium cocoyl isethionate, sodium laureth-5 carboxylate, lauryl ether carboxylic acid, ammonium lauryl sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, potassium lauryl sulfate, potassium laureth sulfate, ammonium cocoyl sulfate, ammonium lauroyl sulfate, sodium cocoyl sulfate, sodium lauroyl sulfate, potassium cocoyl sulfate, monoethanolamine cocoyl sulfate, sodium tridecyl benzene sulfonate, sodium dodecyl benzene sulfonate, sodium C₁₄₋₁₆ olefin sulfonate, sodium lauryl sarcosinate, sodium lauroyl sarcosinate, stearoyl sarcosine, lauryl sarcosine, cocoyl sarcosine, sodium methyl cocoyl taurate, sodium methyl lauroyl taurate, sodium lauroyl glutamate, sodium cocoyl glutamate, disodium cocoyl glutamate, potassium myristoyl glutamate, TEA-cocoyl glutamate, sodium cocoyl glycinate, potassium cocoyl glycinate, sodium cocoyl alaninate, TEA-cocoyl alaninate, or a combination of two or more thereof. For example, the compositions may comprise at least one anionic surfactant chosen from sodium laureth sulfate, sodium lauryl sulfate, sodium lauroyl sulfate, sodium lauroyl methyl isethionate, or a combination of two or more thereof.

If present, the total amount of anionic surfactants may range up to about 15%, such as up to about 12%, up to about 10%, up to about 8%, up to about 5%, up to about 3.5%, or up to about 2% by weight, relative to the total weight of the composition. For example, the total amount of anionic surfactants may range from about 0.01% to about 10%, from about 0.1% to about 8%, from about 0.5% to about 6%, or from about 1% to about 4% by weight, relative to the total weight of the composition. In at least some embodiments, the compositions comprise at least one anionic surfactant, and have a total amount of anionic surfactants ranging from about 0.25% to about 5%, such as from about 0.5% to about 4%, from about 0.75% to about 3%, or from about 1% to about 2% by weight, relative to the total weight of the composition.

### Amphoteric or Zwitterionic Surfactants

In at least some embodiments, the compositions comprise at least one amphoteric or zwitterionic surfactant. Non-limiting examples of useful amphoteric surfactants include derivatives of aliphatic secondary and tertiary amines where the aliphatic radical can be straight or branched chain and one of the aliphatic substituents contains from about 8 to about 18 carbon atoms and one contains an anionic group such as carboxy, sulfonate, sulfate, phosphate, or phosphonate. Exemplary amphoteric surfactants include sodium cocaminopropionate, sodium cocaminodipropionate, sodium cocoamphoacetate, sodium cocoamphohydroxypropylsulfonate, sodium cocoamphopropionate, sodium cornamphopropionate, sodium lauraminopropionate, sodium lauroamphoacetate, sodium lauroamphohydroxypropylsulfonate, sodium lauroamphopropionate, sodium cornamphopropionate, sodium lauriminodipropionate, ammonium cocaminopropionate, ammonium cocaminodipropionate, ammonium cocoamphoacetate, ammonium cocoamphohydroxypropylsulfonate, ammonium cocoamphopropionate, ammonium cornamphopropionate, ammonium lauraminopropionate, ammonium lauroamphoacetate, ammonium lauroamphohydroxypropylsulfonate, ammonium lauroamphopropionate, ammonium cornamphopropionate, ammonium lauriminodipropionate, triethanonlamine cocaminopropionate, triethanonlamine cocaminodipropionate, triethanonlamine cocoamphoacetate, triethanonlamine cocoamphohydroxypropylsulfonate, triethanonlamine cocoamphopropionate, triethanonlamine cornamphopropionate, triethanonlamine lauraminopropionate, triethanonlamine lauroamphoacetate, triethanonlamine lauroamphohydroxypropylsulfonate, triethanonlamine lauroamphopropionate, triethanonlamine cornamphopropionate, triethanonlamine lauriminodipropionate, cocoamphodipropionic acid, disodium caproamphodiacetate, disodium caproamphoadipropionate, disodium capryloamphodiacetate, disodium capryloamphodipriopionate, disodium cocoamphocarboxyethylhydroxypropylsulfonate, disodium cocoamphodiacetate, disodium cocoamphodipropionate, disodium dicarboxyethylcocopropylenediamine, disodium laureth-5 carboxyamphodiacetate, disodium lauriminodipropionate, disodium lauroamphodiacetate, disodium lauroamphodipropionate, disodium oleoamphodipropionate, disodium PPG-2-isodecethyl-7 carboxyamphodiacetate, lauraminopropionic acid, lauroamphodipropionic acid, lauryl aminopropylglycine, and lauryl diethylenediaminoglycine, as well as combinations of two or more thereof.

Betaines may also be used. For example, coco dimethyl carboxymethyl betaine, lauryl dimethyl carboxymethyl betaine, lauryl dimethyl alphacarboxyethyl betaine, cetyl dimethyl carboxymethyl betaine, cetyl dimethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl) alpha-carboxyethyl betaine, coco dimethyl sulfopropyl betaine, stearyl dimethyl sulfopropyl betaine, lauryl dimethyl sulfoethyl betaine, lauryl bis-(2-hydroxyethyl) sulfopropyl betaine, oleyl betaine, cocamidopropyl betaine, or combinations of two or more thereof, may be chosen.

If present, the total amount of amphoteric or zwitterionic surfactants may range up to about 15%, such as up to about 12%, up to about 10%, up to about 8%, up to about 5%, up to about 3.5%, or up to about 2% by weight, relative to the total weight of the composition. For example, the total amount of amphoteric or zwitterionic surfactants may range from about 0.01% to about 10%, from about 0.1% to about 8%, from about 0.5% to about 6%, or from about 1% to about 4% by weight, relative to the total weight of the composition. In at least some embodiments, the compositions comprise at least one amphoteric or zwitterionic surfactant, and have a total amount of amphoteric or zwitterionic surfactants ranging from about 0.25% to about 5%, such as from about 0.5% to about 4%, from about 0.75% to about 3%, or from about 1% to about 2% by weight, relative to the total weight of the composition. In at least some other embodiments, the compositions are free or substantially free of amphoteric surfactants.

### Nonionic Surfactants

The nonionic surfactants may be chosen from alcohols, α-diols and (C₁-C₂₀) alkylphenols, these compounds being polyethoxylated, polypropoxylated and/or polyglycerolated, the number of ethylene oxide and/or propylene oxide groups possibly ranging from 1 to 100, and the number of glycerol groups possibly ranging from 2 to 30, or alternatively these compounds comprising at least one fatty chain comprising from 8 to 30 carbon atoms and especially from 16 to 30 carbon atoms. For example, nonionic surfactants may be chosen from monooxyalkylenated or polyoxyalkylenated (C₈-C₂₄)alkylphenols, saturated or unsaturated, linear or branched, monooxyalkylenated or polyoxyalkylenated C₈-C₃₀ alcohols, saturated or unsaturated, linear or branched, monooxyalkylenated or polyoxyalkylenated C₈-C₃₀ amides, esters of saturated or unsaturated, linear or branched, C₈-C₃₀ acids and of polyalkylene glycols, monooxyalkylenated or polyoxyalkylenated esters of saturated or unsaturated, linear or branched, C₈-C₃₀ acids and of sorbitol, saturated or unsaturated, monooxyalkylenated or polyoxyalkylenated plant oils, condensates of ethylene oxide and/or of propylene oxide, or combinations thereof.

By way of example only, the adducts of ethylene oxide with lauryl alcohol, for example those containing from 9 to 50 oxyethylene units or from 10 to 12 oxyethylene units (Laureth-10 to Laureth-12); the adducts of ethylene oxide with behenyl alcohol, for example those containing from 9 to 50 oxyethylene units (Beheneth-9 to Beheneth-50); the adducts of ethylene oxide with cetearyl alcohol (mixture of cetyl alcohol and stearyl alcohol), for example those containing from 10 to 30 oxyethylene units (Ceteareth-10 to Ceteareth-30); the adducts of ethylene oxide with cetyl alcohol, for example those containing from 10 to 30 oxyethylene units (Ceteth-10 to Ceteth-30); the adducts of ethylene oxide with stearyl alcohol, for example those containing from 10 to 30 oxyethylene units (Steareth-10 to Steareth-30); the adducts of ethylene oxide with isostearyl alcohol, for example those containing from 10 to 50 oxyethylene units (Isosteareth-10 to Isosteareth-50); monoglycerolated or polyglycerolated C₈-C₄₀, e.g. C₈-C₃₀, alcohols, such as lauryl alcohol containing 4 mol of glycerol (Polyglyceryl-4 Lauryl Ether), lauryl alcohol containing 1.5 mol of glycerol, oleyl alcohol containing 4 mol of glycerol (Polyglyceryl-4 Oleyl Ether), oleyl alcohol containing 2 mol of glycerol (Polyglyceryl-2 Oleyl Ether), cetearyl alcohol containing 2 mol of glycerol, cetearyl alcohol containing 6 mol of glycerol, oleocetyl alcohol containing 6 mol of glycerol, or octadecanol containing 6 mol of glycerol; polyoxyethylenated fatty esters such as the adducts of ethylene oxide with esters of lauric acid, palmitic acid, stearic acid or behenic acid, and mixtures thereof, for example those containing from 9 to 100 oxyethylene units such as PEG-9 to PEG-50 laurate, PEG-9 to PEG-50 palmitate, PEG-9 to PEG-50 stearate, PEG-9 to PEG-50 palmitostearate, PEG-9 to PEG-50 behenate, polyethylene glycol 100 EO monostearate (PEG-100 stearate); glyceryl stearate (glyceryl mono-, di- and/or tristearate); glyceryl ricinoleate; sorbitan palmitate; sorbitan isostearate; sorbitan stearate; sorbitan palmitate; sorbitan trioleate; alkylglucose sesquistearates such as methylglucose sesquistearatel alkylglucose palmitates such as methylglucose or ethylglucose palmitate, etc., or combinations of two or more thereof may be chosen.

If present, the total amount of nonionic surfactants may range up to about 15%, such as up to about 12%, up to about 10%, up to about 8%, up to about 5%, up to about 3.5%, or up to about 2% by weight, relative to the total weight of the composition. For example, the total amount of nonionic surfactants may range from about 0.01% to about 10%, from about 0.1% to about 8%, from about 0.5% to about 6%, or from about 1% to about 4% by weight, relative to the total weight of the composition. In at least some embodiments, the compositions comprise at least one nonionic surfactant, and have a total amount of nonionic surfactants ranging from about 0.25% to about 5%, such as from about 0.5% to about 4%, from about 0.75% to about 3%, or from about 1% to about 2% by weight, relative to the total weight of the composition. In at least some embodiments, the compositions are free or substantially free of nonionic surfactants.

In some embodiments, compositions according to the disclosure are free or substantially free of cationic surfactants.

### Fatty Compounds

Optionally, compositions according to the disclosure may include at least one fatty compound. In certain embodiments, the at least one fatty compound may be chosen from lower alkanes, fatty alcohols, fatty acids, esters of fatty acids, esters of fatty alcohols, oils such as mineral, vegetable, animal, silicone and non-silicone oils, silicone and non-silicone waxes, or combinations of any two or more thereof. In some embodiments, the compositions comprise at least one fatty compound of natural origin. In some embodiments, the compositions are free of fatty compounds that are not of natural origin.

Non-limiting examples of silicone oils include dimethicone, amodimethicone, cyclomethicone, polysilicone-11, phenyl trimethicone, trimethylsilylamodimethicone, and stearoxytrimethylsilane. For example, the composition may comprise at least one silicone chosen from amodimethicone, PEG-7 Dimethicone, PEG-8 Dimethicone, PEG-9 Dimethicone, PEG-10 Dimethicone, PEG-12 Dimethicone, PEG-14 Dimethicone, PEG-17 Dimethicone, PEG/PPG-3/10 Dimethicone, PEG/PPG-4/12 Dimethicone, PEG/PPG-17/18 Dimethicone, cetyl PEG/PPG-10/1 dimethicone, Dimethicone PEG-8 Benzoate, Dimethicone PEG-7 Phosphate, Dimethicone PEG-8 Phosphate, Dimethicone PEG-10 Phosphate, or a combination of two or more thereof. In some preferred embodiments, the compositions comprise a silicone oil component that comprises, consists essentially of, or consists of dimethicone, amodimethicone, or a combination thereof. In some embodiments, the compositions are free or substantially free of silicone oils and/or silicone waxes.

In some embodiments, compositions according to the disclosure may include at least one fatty compound chosen from fatty alcohols. In certain embodiments, “fatty alcohol” refers to any alcohol with a carbon chain of C5 or greater, such as, for example, C8 or greater, C10 or greater, or C12 or greater, such as from 6 to 30 carbon atoms or from 8 to 30 carbon atoms. The fatty alcohols may be alkoxylated or non-alkoxylated, saturated or unsaturated, and linear or branched. Non-limiting examples of fatty alcohols include arachidyl alcohol, behenyl alcohol, caprylic alcohol, cetearyl alcohol, cetyl alcohol, coconut alcohol, decyl alcohol, hydrogenated tallow alcohol, jojoba alcohol, lauryl alcohol, myristyl alcohol, oleyl alcohol, palm alcohol, palm kernel alcohol, stearyl alcohol, tallow alcohol, tridecyl alcohol, or combinations of two or more thereof. In some preferred embodiments, the compositions comprise a fatty alcohol component that comprises, consists essentially of, or consists of cetyl alcohol, stearyl alcohol, cetearyl alcohol, or combinations thereof.

In some embodiments, the composition may include one or more fatty compounds chosen from oils of animal, vegetable, or mineral origin (e.g. lanolin, squalene, fish oil, perhydrosqualene, mink oil, turtle oil, soybean oil, grape seed oil, sesame oil, maize oil, rapeseed oil, sunflower oil, cottonseed oil, avocado oil, olive oil, castor seed oil, jojoba seed oil, peanut oil, sweet almond oil, palm oil, cucumber oil, hazelnut oil, apricot kernel oil, wheat germ oil, calophyllum oil, macadamia oil, coconut oil, cereal germ oil, candlenut oil, thistle oil, candelilla oil, safflower oil, or shea butter), linear or branched hydrocarbons (e.g. polybutene, hydrogenated polyisobutene, polyisoprene, polydecenes such as hydrogenated polydecene, or also linear, branched and/or cyclic alkanes which are optionally volatile, such as, for example, isohexadecane, isododecane, isodecane, or isohexadecane), mono- and/or polyesters of fatty acids and/or of fatty alcohols (e.g. mono- and polyesters of hydroxy acids and of fatty alcohols, esters of benzoic acid and of fatty alcohols, polyesters of polyols, dipentaerythrityl C5-C9 esters, trimethylolpropane polyesters, propylene glycol polyesters, or polyesters of hydrogenated castor oil), perfluorinated and/or organofluorinated oils, fluorosilicone oils, or combinations of two or more thereof. Non-limiting examples of fatty acids include optionally branched and/or unsaturated fatty acids such as myristic acid, palmitic acid, stearic acid, behenic acid, oleic acid, linoleic acid, linolenic acid, isostearic acid, or combinations of two or more thereof.

If present, the total amount of fatty compounds in the composition may range up to about 20%, such as up to about 18%, up to about 15%, up to about 12%, up to about 10%, up to about 8%, or up to about 5% by weight, relative to the total weight of the composition. For example, the total amount of fatty compounds may range from about 0.1% to about 20%, such as from about 0.5% to about 18%, from about 1% to about 15%, from about 1.25% to about 12%, or from about 1.5% to about 10% by weight, relative to the total weight of the composition. In at least some embodiments, the compositions are free or essentially free of fatty compounds.

### Thickening Agents

Compositions according to the disclosure optionally comprise at least one thickening agent. Useful thickening agents include, but are not limited to, semisynthetic polymers, such as semisynthetic cellulose derivatives, synthetic polymers, such as carbomers, poloxamers, and acrylates/beheneth-25 methacrylate copolymer, acrylates copolymer, polyethyleneimines (e.g., PEI-10), naturally occurring polymers, such as acacia, tragacanth, alginates (e.g., sodium alginate), carrageenan, vegetable gums, such as xanthan gum, guar gum, petroleum jelly, waxes, particulate associate colloids, such as bentonite, colloidal silicon dioxide, and microcrystalline cellulose, celluloses such as hydroxyethylcellulose and hydroxypropylcellulose, and guars such as hydroxypropyl guar.

In some embodiments, the thickening agent may be chosen from associative thickening polymers such as anionic associative polymers, amphoteric associative polymers, cationic associative polymers, or nonionic associative polymers. A non-limiting example of an amphoteric associative polymer is acrylates/beheneth-25 methacrylate copolymer, and non-limiting examples of anionic associative polymers include acrylates copolymer and acrylates crosspolymer-4.

If present, the total amount of thickening agents may range from about 0.001% to about 5%, such as from about 0.01% to about 4%, from about 0.1% to about 3.5%, from about 0.2% to about 3%, from about 0.3% to about 2.5% by weight, from about 0.4% to about 2%, from about 0.5% to about 1.5%, or from about 0.5% to about 1%, relative to the total weight of the composition.

### Carboxylic Acids

Compositions according to the disclosure optionally comprise at least one carboxylic acid. In some embodiments, the compositions comprise more than one carboxylic acid, for example, at least two carboxylic acids, at least three carboxylic acids, etc. For example, mono-, di-, or tri-carboxylic acids may be chosen.

In various embodiments, the at least one carboxylic acid may be chosen from citric acid, maleic acid, succinic acid, aspartic acid, glutamic acid, lactic acid, malic acid, tartaric acid, salts thereof, or combinations thereof, preferably citric acid, lactic acid, salts thereof, or combinations thereof. In one embodiment, citric acid may be chosen.

If present, the total amount of carboxylic acids may range up to about 3%, such as up to about 2.5%, up to about 2%, up to about 1.5%, up to about 1%, or up to about 0.5% by weight, relative to the total weight of the composition. For example, the total amount of carboxylic acids may range from about 0.001% to about 3%, from about 0.01% to about 2.5%, from about 0.1% to about 2%, or from about 0.1% to about 1.5% by weight, relative to the total weight of the composition. In some embodiments, the total amount of mono-, di-, and tri-carboxylic acids does not exceed about 1%, for example may range from 0.001% to about 1%, such as from about 0.001% to about 0.9%, from about 0.001% to about 0.8%, from about 0.001% to about 0.7%, from about 0.001% to about 0.6%, from about 0.001% to about 0.5%, from about 0.001% to about 0.4%, from about 0.001% to about 0.3%, from about 0.001% to about 0.2%, or from about 0.001% to about 0.1% by weight, relative to the total weight of the composition.

In some embodiments, the compositions are free or essentially free of carboxylic acids, or are free or essentially free of mono-, di-, and/or tri-carboxylic acids. In other embodiments, the compositions are free or essentially free of citric acid.

### Auxiliary Components

Compositions according to the disclosure may optionally include one or more auxiliary components. Non-limiting examples include preservatives, fragrances, pH adjusters (e.g. citric acid, maleic acid, malic acid, malonic acid, etc.), salts, antioxidants, vitamins (e.g. ascorbic acid, tocopherol), vitamin derivatives, botanical extracts, buffers, sequestering agents, and the like.

The total amount of auxiliary components, if present, typically ranges from about 0.01% to about 15% by weight, based on the total weight of the composition. For example, in some embodiments the individual amounts of each component or the total amount of components may range from about 0.1% to about 10%, about 0.1% to about 8%, about 0.1% to about 5%, about 0.1% to about 4%, about 0.1% to about 3%, about 0.1% to about 2%, about 0.25% to about 10%, about 0.25% to about 8%, about 0.25% to about 5%, about 0.25% to about 4%, about 0.25% to about 3%, about 0.25% to about 2%, about 0.5% to about 10%, about 0.5% to about 8%, about 0.5% to about 5%, about 0.5% to about 4%, about 0.5% to about 3%, about 0.5% to about 2%, about 0.75% to about 10%, about 0.75% to about 8%, about 0.75% to about 5%, about 0.75% to about 4%, about 0.75% to about 3%, or about 0.75% to about 2% by weight, based on the total weight of the composition.

In some embodiments, the compositions may be free of sulfites, bisulfites, metabisulfites, persulfates, peroxides, and/or ammonia. In yet other embodiments, the compositions may be essentially free of sulfites, bisulfites, metabisulfites, persulfates, peroxides, and/or ammonia, for example may comprise less than 5%, less than 4.5%, less than 4%, less than 3.5%, less than 3%, less than 2.5%, less than 2%, less than 1.5%, less than 1.25%, less than 1%, less than 0.75%, less than 0.5%, less than 0.4%, less than 0.3%, less than 0.2%, less than 0.1%, less than 0.05%, or less than 0.01% of a total amount of sulfites, bisulfites, metabisulfites, persulfates, peroxides, or ammonia, or of a total amount of any combination of two or more thereof.

The compositions may be in any suitable form. For example, the compositions may be a liquid, a gel, a gel cream, a cream, a serum, etc. Advantageously, the compositions can be used as a one-part hair color removing composition, as compared to known systems for removing color from hair that require the application of multiple compositions to effectively remove color from the hair.

The pH of the composition is typically acidic, i.e. below 7, such as below about 6, below about 5, or below about 4. For example, compositions according to the disclosure may have a pH ranging from about 1 to about 6, such as from about 2 to about 5. In some embodiments, the compositions have a pH ranging from about 2.5 to about 5, from about 2.75 to about 4.75, or from about 3 to about 4.5.

### II. Methods

The disclosure further relates to methods of removing color from keratin fibers, especially hair that has been previously dyed such as with an oxidation dye. The methods comprise applying a composition according to the disclosure onto the keratin fibers, e.g. hair, optionally leaving the composition on the keratin fibers for a period of time (“rest period,” “resting period,” or “leave-in period”), and subsequently rinsing the composition from the keratin fibers. Thus, the methods may comprise a two-step method, which is simpler and more effective than known three- or more step methods where multiple compositions are applied to the hair for color removal before the hair is rinsed.

The appropriate leave-in period will be determined based on the color to be removed, and may in various embodiments last up to about 1 minute, up to about 2 minutes, up to about 5 minutes, up to about 10 minutes, up to about 20 minutes, up to abut 30 minutes, up to about 45 minutes, up to about 1 hour, up to about 2 hours, etc., such as from about 1 minute to about 60 minutes, from about 2 minutes to about 45 minutes, from about 5 minutes to about 40 minutes, or from about 10 minutes to about 30 minutes.

Surprisingly, the compositions and methods according to the disclosure effectively remove color imparted to hair by previously-applied oxidative dyes without the use of sulfites, bisulfites, metabisulfites, persulfates, peroxides, and/or ammonia, and can prevent or substantially prevent the oxidative dyes from re-oxidizing such that the color does not return. Thus, in at least some embodiments, the compositions are free or essentially free of sulfites, bisulfites, metabisulfites, persulfates, peroxides, and/or ammonia, and/or the methods include only compositions that are free or substantially free of sulfites, bisulfites, metabisulfites, persulfates, peroxides, and/or ammonia. This benefit has surprisingly been observed on hair even several months after the original color was removed with compositions and methods according to the disclosure, and thus the color removal is considered to be permanent, which is a benefit not achieved by traditional hair color removing compositions that use reducing agents.

In addition, the synergistic combination of components in compositions and methods according to the disclosure also surprisingly reduces or eliminates the odor that is typically encountered with compositions and methods for removing color from hair.

Having described the many embodiments of the present invention in detail, it will be apparent that modifications and variations are possible without departing from the scope of the disclosure defined in the appended claims. Furthermore, it should be appreciated that all examples in the present disclosure, while illustrating many embodiments of the disclosure, are provided as non-limiting examples and are, therefore, not to be taken as limiting the various aspects so illustrated. It is to be understood that all definitions herein are provided for the present disclosure only.

As used herein, the terms “comprising,” “having,” and “including” (or “comprise,” “have,” and “include”) are used in their open, non-limiting sense.

In this application, the use of the singular includes the plural unless specifically stated otherwise. The singular forms “a,” “an,” “the,” and “at least one” are understood to encompass the plural as well as the singular unless the context clearly dictates otherwise. The expression “one or more” and “at least one” are interchangeable and expressly include individual components as well as mixtures/combinations. Likewise, the term “a salt thereof” also relates to “salts thereof.” Thus, where the disclosure refers to “at least one element selected from the group consisting of A, B, C, D, E, F, a salt thereof, or mixtures thereof,” it indicates that that one or more of A, B, C, D, and F may be included, one or more of a salt of A, a salt of B, a salt of C, a salt of D, a salt of E, and a salt of F may be included, or a mixture of any two or more of A, B, C, D, E, F, one or more salts of A, one or more salts of B, one or more salts of C, one or more salts of D, one or more salts of E, and one or more salts of F may be included.

The term “and/or” should be understood to include both the conjunctive and the disjunctive. For example, “water and/or non-aqueous solvents” means “water and non-aqueous solvents” as well as “water or non-aqueous solvents,” and expressly covers instances of either.

As used herein, the phrases “and mixtures thereof,” “and a mixture thereof,” “and combinations thereof,” “and a combination thereof,” “or mixtures thereof,” “or a mixture thereof,” “or combinations thereof,” and “or a combination thereof,” are used interchangeably to denote that the listing of components immediately preceding the phrase, such as “A, B, C, D, or mixtures thereof” signify that the component(s) may be chosen from A, from B, from C, from D, from A+B, from A+B+C, from A+D, from A+C+D, etc., without limitation on the variations thereof. Thus, the components may be used individually or in any combination thereof.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

All ranges and amounts given herein are intended to include sub-ranges and amounts using any disclosed point as an end point, and all endpoints are intended to be included unless expressly stated otherwise. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not expressly stated, unless expressly stated otherwise. Similarly, a range given of “about 1% to 10%” is intended to have the term “about” modifying both the 1% and the 10% endpoints. The term “about” is used herein to indicate a difference of up to +/−10% from the stated number, such as +/−9%, +/−8%, +/−7%, +/−6%, +/−5%, +/−4%, +/−3%, +/−2%, or +/−1%. Likewise, all endpoints of ranges are understood to be individually disclosed, such that, for example, a range of 1:2 to 2:1 is understood to disclose a ratio of both 1:2 and 2:1.

As used herein, if a component is described as being present “in an amount up to” a certain amount, it is intended that such component is, in fact, present in the composition, i.e. is present in an amount greater than 0%.

All amounts and ratios herein are given based upon the total weight of the composition, unless otherwise indicated. Unless otherwise indicated, all percentages herein are by weight of active material.

As used herein, the phrase “applying a composition onto keratin fibers” and variations thereof are intended to mean contacting the keratin fibers such as hair with at least one of the compositions of the disclosure, in any manner. It may also mean contacting the keratin fibers with an effective amount of the composition.

As used herein, the term “salts” referred to throughout the disclosure may include salts having a counterion such as an alkali metal, alkaline earth metal, or ammonium counterion. This list of counterions, however, is non-limiting. Salts also include a dissociated form of a compound, e.g. in an aqueous solution.

As used herein, the term “substantially free” or “essentially free” means the specific material may be present in small amounts that do not materially affect the basic and novel characteristics of the compositions according to the disclosure. For instance, there may be less than 2% by weight of a specific material added to a composition, based on the total weight of the compositions (provided that an amount of less than 2% by weight does not materially affect the basic and novel characteristics of the compositions according to the disclosure. Similarly, the compositions may include less than 2%, less than 1.5%, less than 1%, less than 0.5%, less than 0.1%, less than 0.05%, or less than 0.01%, or none of the specified material. Furthermore, all components that are positively set forth in the instant disclosure may be negatively excluded from the claims, e.g., a claimed composition may be “free,” “essentially free” (or “substantially free”) of one or more components that are positively set forth in the instant disclosure. The term “substantially free” or “essentially free” as used herein may also mean that the specific material is not added to the composition but may still be present in a raw material that is included in the composition.

As used herein, the terms “treat,” “treated,” “treatment,” and variations thereof is not intended to be limiting, but rather is merely intended to indicate that one or more compositions is applied to the hair, and optionally removed from the hair. For example, hair that is “treated” with a composition according to the disclosure may have had the composition applied, and/or may have had the composition applied and removed, e.g. by rinsing or towel drying. As a further example, hair that is “treated” with a composition according to the disclosure may have had the composition applied, and/or may have had the composition applied and rinsed from the hair.

For purposes of the disclosure, the terms “color removal,” “hair color-removing,” and variations thereof should be understood to refer to removal of artificial color from the hair imparted by previously-applied oxidative dyes. It should be understood that removal of color can include removal of all color, or can include removal of various tones of color. Therefore, in some embodiments, overall lightening of the hair color will be observed (e.g. greater ΔE), while in other embodiments even if the overall color change is not considered significant, variation in one or more of the L*, a*, or b* values will be observed.

The examples that follow serve to illustrate embodiments of the present disclosure without, however, being limiting in nature. It will be apparent to those skilled in the art that various modifications and variations can be made in the compositions and methods of the invention without departing from the spirit or scope of the invention.

### EXAMPLES

The following Examples are intended to be non-limiting and explanatory in nature only. In the Examples, amounts are expressed in percentage by weight (wt %) of active materials, relative to the total weight of the composition, unless otherwise indicated.

In these Examples, the change in the color of hair is evaluated with the CIE L* a* b* system, using Colorshot MS, where the change is determined by evaluating the color of the hair after treatment (L*₂, a*₂, b*₂) compared to the color of the hair before treatment (L*₁, a*₁, b*₁). The change in color (ΔE) is defined as:

ΔE*_{ab}=\sqrt{(L*₂−L*₁)²+(a*₂−a*₁)²+(b*₂−b*₁)²}

In this system, the three parameters represent, respectively, the color intensity (L*), the green/red color axis (a*) and the blue/yellow color axis (b*). The higher the value of L*, the lighter the color, the higher the value of a*, the redder the color, and the higher the value of b*, the yellower the color. The higher the value for ΔE, the greater the difference in color of treated hair relative to the color of the hair prior to treatment.

### Example 1—Compositions

Compositions 1A-1E according to the disclosure and comparative composition C1 were prepared as shown in Table 1. The pH of each composition was about 3.5.

TABLE 1

| | 1A | 1B | 1C | 1D | 1E | C1 |
|---|---|---|---|---|---|---|
| Thioglycolic acid | 3.0 | | | | | |
| Thiolactic acid | | 5.0 | | | | |
| Glycolic acid | | | 7.0 | 7.0 | | |
| a-ketoglutaric acid | 10 | 10 | 10 | 10 | 10 | |
| Betaine | 1.6 | | 1.6 | | 1.6 | |
| Arginine | 2.5 | 3.0 | 2.5 | | | |
| Glycine | | | | 5.0 | | |
| Ethanolamine | 3.7 | 1.7 | | | | |
| Triethanolamine | | | | | 15 | |
| Bentonite | 10 | 5.0 | 10 | | | |
| Xanthan gum | 0.7 | 0.7 | 0.7 | | 0.7 | 0.7 |
| Citric acid | | | | | | 5.0 |
| Sodium lauryl sulfate | 1.5 | 1.5 | 1.5 | | 1.5 | |
| Sodium lauroyl methyl | | | | | | 1.7 |
| isethionate | | | | | | |
| Water | QS to 100 | QS to 100 | QS to 100 | QS to 100 | QS to 100 | QS to 100 |

### Example 2—Evaluation of Color Removal

Six swatches of permed, ˜90% grey hair were colored with a commercially-available oxidative hair dye composition. The oxidative hair dye composition was mixed in a 1:1 ratio with 20V hydrogen peroxide developer to form a hair dye mixture, and the hair dye mixture was applied to the swatches at a rate of about 3 grams per gram of hair. The swatches were left to process at room temperature for about 35 minutes, rinsed, shampooed with a commercial shampoo, rinsed again, and allowed to air dry.

Once the swatches were dry, the swatches were treated as follows. Each of compositions 1A-1D and C1 were applied to separate swatches at a rate of about 2 grams per gram of hair. The swatches were allowed to rest for 30 minutes at 33° C. The swatches were then rinsed for about 30 seconds, shampooed with a commercial shampoo, rinsed, shampooed again, rinsed again, and then blow-dried.

One swatch colored with the hair dye mixture was not treated with a color-removing composition, and was used as a control.

The color of each of the swatches was then evaluated with the CIE L* a* b* system, as described above.

First, the color of each of the swatches was evaluated immediately after the swatches were treated as described above and dried (time=T₀), to evaluate the effectiveness of color removal. Table 2A shows the L*, a*, b* and ΔE values at T₀.

TABLE 2A

| | | L* | a* | b* | ΔE |
|---|---|---|---|---|---|
| L*₁, a*₁, b*₁ | CONTROL | 5.90 | 1.64 | 0.20 | — |
| L*₂, a*₂, b*₂ | 1A | 49.19 | 8.92 | 25.10 | 50.47 |
| | 1B | 43.90 | 8.25 | 24.68 | 45.68 |
| | 1C | 36.82 | 7.86 | 17.11 | 35.78 |
| | 1D | 28.66 | 8.53 | 9.68 | 25.60 |
| | C1 | 7.53 | 0.04 | 0.74 | 2.35 |

Table 2A demonstrates that compositions according to the disclosure are surprisingly more effective for removing color from hair compared to compositions not according to the disclosure, as demonstrated by the significantly higher ΔE values for swatches treated with compositions 1A-1D compared to the swatch treated with composition C1. Since a difference in ΔE of ˜2 is visible to the naked eye, these results are significant.

The color of the swatches treated with compositions 1A-1C and C1 was again evaluated after one week (time=T₇), to evaluate the lastingness of the color removal. Table 2B shows the L*, a*, b* and ΔE values at T₇.

TABLE 2B

| | | L* | a* | b* | ΔE |
|---|---|---|---|---|---|
| L*₁, a*₁, b*₁ | CONTROL | 5.90 | 1.64 | 0.20 | — |
| L*₂, a*₂, b*₂ | 1A | 46.78 | 8.02 | 23.18 | 47.33 |
| | 1B | 44.68 | 7.66 | 23.35 | 45.56 |
| | 1C | 39.92 | 7.11 | 21.13 | 40.32 |
| | C1 | 7.53 | 0.04 | 0.74 | 2.35 |

Although the L*, a*, b* and ΔE values for the swatches treated with compositions 1A-1C are slightly higher or lower in Table 2B than in Table 2A, these differences are considered to be within an acceptable deviation. Visually, there was no difference in color of the swatches at T₇ compared to T₀.

Tables 2A and 2B therefore demonstrate that color removal with compositions according to the disclosure is not only highly effective, but also that the color removal lasts through at least one week. Additionally, it was observed that even after several months, the swatches treated with compositions 1A-1D visually appeared to maintain the same color, demonstrating that re-oxidation of the color does not occur.

### Example 3—Compositions Including Amines

Compositions 3A-3C according to the disclosure were prepared as shown in Table 3. The pH of each composition was about 3.5.

TABLE 3

| | 3A | 3B | 3C |
|---|---|---|---|
| Glycolic acid | 7.0 | 7.0 | 7.0 |
| Glycine | 5.0 | 5.0 | 5.0 |
| Monoethanolamine | | 5.7 | |
| Triethanolamine | 15 | | |
| Aminomethylpropanol | | | 8.0 |
| Water | QS to 100 | QS to 100 | QS to 100 |

### Example 4—Evaluation of Color Removal

Three swatches of virgin, ˜90% grey hair, and four swatches of permed, ˜90% grey hair (7 swatches total) were colored with a commercially-available oxidative hair dye composition. The oxidative hair dye composition was mixed in a 1:1 ratio with 20V hydrogen peroxide developer to form a hair dye mixture, and the hair dye mixture was applied to the swatches at a rate of about 3 grams per gram of hair. The swatches were left to process at room temperature for about 35 minutes, rinsed, shampooed with a commercial shampoo, rinsed again, and allowed to air dry.

Once the swatches were dry, the swatches were treated as follows. Each of compositions 3B and 3C were applied to separate virgin swatches, and each of compositions 3A-3C were applied to separate permed swatches, at a rate of about 2 grams per gram of hair, so that the color removing ability of each composition could be evaluated on both virgin hair colored with the hair dye mixture and permed hair colored with the hair dye mixture. The swatches were allowed to rest for 30 minutes at 33° C. The swatches were then rinsed for about 30 seconds, shampooed with a commercial shampoo, rinsed, shampooed again, rinsed again, and then blow-dried.

One virgin and one permed swatch colored with the hair dye mixture but not treated with a color-removing composition were used as controls.

The color of each of the swatches was evaluated with the CIE L* a* b* system immediately after the swatches were treated as described above and dried, to evaluate the effectiveness of color removal. Tables 4A-4B show the L*, a*, b* and ΔE values for the virgin (Table 4A) and permed (Table 4B) swatches.

TABLE 4A

| | | L* | a* | b* | ΔE |
|---|---|---|---|---|---|
| L*₁, a*₁, b*₁ | CONTROL | 5.90 | 1.64 | 0.20 | — |
| L*₂, a*₂, b*₂ | 3B | 17.96 | 6.28 | 5.91 | 14.13 |
| | 3C | 6.07 | 1.91 | −0.12 | 0.45 |

TABLE 4B

| | | L* | a* | b* | ΔE |
|---|---|---|---|---|---|
| L*₁, a*₁, b*₁ | CONTROL | 5.90 | 1.64 | 0.20 | — |
| L*₂, a*₂, b*₂ | 3A | 28.66 | 8.53 | 9.68 | 25.60 |
| | 3B | 34.71 | 8.94 | 15.80 | 33.56 |
| | 3C | 30.74 | 8.73 | 13.70 | 29.14 |

The data in Tables 4A-4B demonstrate that the addition of various amines can improve the color-removal properties of reducing agents according to the disclosure, particularly on hair that has previously been chemically treated. In addition, monoethanolamine surprisingly provides the greatest improvement in color removal, even compared to other amine compounds.

### Example 5—Additional Compositions

Compositions 5A-5D according to the disclosure can be prepared as shown in Table 5, and are likewise expected to provide synergistic hair color removing benefits.

TABLE 5

| | 5A | 5B | 5C | 5D |
|---|---|---|---|---|
| Thioglycolic acid | | 15 | | |
| Thiolactic acid | | | 9 | |
| Glyoxylic acid | 3 | | | |
| Glutaric acid | | | | 12 |
| Proline | 1 | | 4 | |
| Betaine | | 5 | | 6 |
| Arginine | | 5 | | |
| Hydroxypropyl guar | 1 | 0.5 | 1.5 | 2 |
| Sodium lauroyl sulfate | 2 | 2.5 | 1.5 | 1 |
| Additives (pH adjuster, | ≤3 | ≤3 | ≤3 | ≤3 |
| preservative, fragrance) | | | | |
| Solvent | QS to 100 | QS to 100 | QS to 100 | QS to 100 |

The above Examples confirm that compositions and methods according to the disclosure surprisingly and unexpectedly enhance removal of color from hair, compared to compositions and methods not according to the disclosure.

1. A hair-color removing composition comprising:
(a) at least one reducing agent chosen from glutaric acid, glyoxylic acid, thiolactic acid, thioglycolic acid, glycolic acid, citric acid, or combinations of two or more thereof;
(b) at least one amino acid;
(c) optionally, at least one amine compound; and
(d) at least one solvent.

2. The hair-color removing composition of claim 1, wherein the total amount of reducing agents (a) ranges from about 1% to about 20% by weight, relative to the total weight of the composition.

3. The hair-color removing composition of claim 1, wherein the reducing agent (a) comprises α-ketoglutaric acid, and optionally further comprises at least one additional reducing agent chosen from glutaric acid compounds other than α-ketoglutaric acid, glyoxylic acid, thiolactic acid, thioglycolic acid, glycolic acid, and/or citric acid.

4. The hair-color removing composition of claim 1, wherein the total amount of amino acids (b) ranges from about 0.1% to about 10% by weight, relative to the total weight of the composition.

5. The hair-color removing composition of claim 1, comprising at least one amino acid chosen from arginine, glycine, betaine, proline, or combinations of two or more thereof.

6. The hair-color removing composition of claim 1, wherein the total amount of amine compounds (c) ranges from about 0.1% to about 20% by weight, relative to the total weight of the composition.

7. The hair-color removing composition of claim 1, comprising monoethanolamine in an amount ranging from about 1% to about 10% by weight, relative to the total weight of the composition.

8. The hair-color removing composition of claim 1, further comprising at least one component chosen from thickening agents, fatty compounds, clay compounds, carboxylic acids, anionic surfactants, non-ionic surfactants, amphoteric/zwitterionic surfactants, or combinations of two or more thereof.

9. The hair-color removing composition of claim 1, wherein the composition is free of reducing agents other than glutaric acid, glyoxylic acid, thiolactic acid, thioglycolic acid, glycolic acid, and/or citric acid.

10. A hair-color removing composition comprising:
(a) at least one reducing agent chosen from glutaric acid, glyoxylic acid, thiolactic acid, thioglycolic acid, glycolic acid, citric acid, or combinations of two or more thereof;
(b) at least one amino acid;
(c) monoethanolamine; and
(d) water,
wherein the pH of the composition ranges from about 1 to about 6.

11. The hair-color removing composition of claim 10, wherein the total amount of reducing agents (a) ranges from about 1% to about 20% by weight, relative to the total weight of the composition.

12. The hair-color removing composition of claim 10, wherein the reducing agent (a) comprises α-ketoglutaric acid, and optionally further comprises at least one additional reducing agent chosen from glutaric acid compounds other than α-ketoglutaric acid, glyoxylic acid, thiolactic acid, thioglycolic acid, glycolic acid, and/or citric acid.

13. The hair-color removing composition of claim 10, wherein the total amount of amino acids (b) ranges from about 0.1% to about 10% by weight, relative to the total weight of the composition.

14. The hair-color removing composition of claim 10, comprising at least one amino acid chosen from arginine, glycine, betaine, proline, or combinations of two or more thereof.

15. The hair-color removing composition of claim 10, wherein the total amount of monoethanolamine ranges from about 1% to about 15% by weight, relative to the total weight of the composition.

16. A method for removing color from hair, the method comprising:
i. applying to the hair a hair-color removing composition comprising:
a. at least one reducing agent chosen from glutaric acid, glyoxylic acid, thiolactic acid, thioglycolic acid, glycolic acid, citric acid, or combinations of two or more thereof;
b. at least one amino acid;
c. optionally, at least one amine compound; and
d. at least one solvent, and
ii. rinsing the hair.

17. The method according to claim 16, wherein the hair-color removing composition comprises a total amount of reducing agents (a) ranging from about 1% to about 20% by weight, relative to the total weight of the composition.

18. The method according to claim 16, wherein the hair-color removing composition comprises a total amount of amino acids (b) ranging from about 0.1% to about 10% by weight, relative to the total weight of the composition.

19. The method according to claim 16, wherein the hair-color removing composition comprises a total amount of amine compounds (c) ranging from about 0.1% to about 20% by weight, relative to the total weight of the composition.

20. The method according to claim 16, wherein the hair to which the hair-color removing composition is applied comprises at least one oxidative dye.
